(19) United States
(12) Patent Application Publication
(10) Pub. No.: US 2015/0376581 A1
(43) Pub. Date: Dec. 31, 2015

(54) T7 RNA POLYMERASE VARIANTS AND METHODS OF USING THE SAME

(71) Applicant: TECHNISCHE UNIVERSITÄT DORTMUND, Dortmund (DE)

(72) Inventors: Susanne BRAKMANN, Dortmund (DE); Jenny IBACH, Dortmund (DE)

(73) Assignee: Technische Universitaet Dortmund, Dortmund (DE)

(21) Appl. No.: 14/439,133

(22) PCT Filed: Oct. 29, 2012
(86) PCT No.: PCT/EP2012/071396

§ 371 Date: Sep. 21, 2015

(51) Int. Cl. C12N 9/12; C12P 19/34

(52) U.S. Cl. CPC C12N 9/1247; C12P 19/34; C12Y 207/07007

(57) ABSTRACT

The present invention relates to T7 RNA polymerase variants with improved affinity for 2′-modified nucleotides compared to the wildtype as well as methods for their production and methods of using them. The present invention also relates to the 2′-modified RNA molecules produced according to the methods of the invention.

### FIELD OF THE INVENTION

The present invention lies in the field of molecular biology and relates to T7 RNA polymerase variants with improved affinity for 2′-modified nucleotides compared to the wildtype as well as methods for their production and methods of using them. The present invention also relates to the 2′-modified RNA molecules produced according to the methods of the invention.

### BACKGROUND OF THE INVENTION

RNA not only is a central player in mobilizing and interpreting genetic information, it also exhibits various regulating or, directing cellular functions due to its ability to adopt a wide variety of conformations. Some RNAs fold to form catalytic centers while others show structures that operate via specific binding interactions to RNA, DNA, or proteins.

These findings supported ideas to exploit RNA molecules as therapeutic agents to combat a variety of human diseases. A major obstacle in employing RNA molecules in therapeutic applications however is their poor stability in vivo. RNA intended for clinical application is thus usually modified in order to increase its otherwise poor resistance to cellular nucleases and to optimize its pharmacokinetic profile, i.e., the extent and rate of its liberation, absorption, distribution, metabolism and excretion in an organism. Chemical modification of backbone or side chains of the nucleic acids can for example significantly improve the efficacy of RNA therapeutics while retaining conformational characteristics and function of the unmodified molecule.

Substitutions of the ribose 2′ hydroxyl moiety by either fluoro or O-methyl (O-me) groups are the most typical modifications. RNA aptamers that contain all 2′-O-me-modified nucleotides are extremely resistant to chemical, physical, thermal and enzymatic damage and bind their target molecule even after 25 minutes of autoclaving at a peak temperature of 125° C. {P. E. Burmeister et al. (2005) Chemistry & Biology, 12, 25}. In vivo, the same aptamers show increased nuclease resistance leading to clearance half-lives of 23 h (in mice) that compare favorably with nuclease-susceptible, unmodified RNAs exhibiting half-lives of less than 1 h.

In vitro, completely 2% O-me-modified RNAs do not act as substrates for Taq DNA polymerase and, thus, cannot be amplified during PCR while they are copied on a limited scale by reverse transcriptase. Enzymatic synthesis of 2% O-me-modified RNAs is however preferred, especially with respect to the SELEX procedure for identifying RNA aptamers.

Wildtype T7 RNA polymerase, the enzyme that is commonly used in these processes, however, is inefficient in incorporating modified nucleotides. Mutant enzymes have been engineered that promote the incorporation of 2′-modified nucleotides: Burmeister et al. employed T7 RNA polymerase variant Y639F/H784A {R. Padilla, R. Sousa (2002) Nucleic Acids Res., 30, e138} and optimized reaction conditions {P. E. Burmeister et al. (2005) Chemistry & Biology, 12, 25} for generating 2% O-me-modified transcripts, and Chelliserrykattil and Ellington used a combined selection/screening procedure for the identification of another variant, E593GN685A, which was shown to incorporate all 2′-O-me nucleotides except 2′-O-me GTP as well as combinations of the three modified nucleotides {J. Chelliserrykattil, A. D. Ellington (2004) Nature Biotechnol., 22, 1155}.

Still, transcription by mutant T7 RNA polymerases incorporating 2′-O-me-modified nucleotides cannot compare to the reaction with natural nucleotides as it does hardly involve any amplification and also, shows significantly reduced processivity. Neither of the RNA polymerases studied so far can be employed for the generation of fully 2% O-me-modified transcripts that are long enough to contain aptamers.

Hence, there is need in the art for materials and methods that allow improving the synthesis of 2′-methoxy modified RNA molecules, for example with respect to yields and processivity.

### SUMMARY OF THE INVENTION

The present invention is based on the inventors' finding that variants of T7 RNA polymerase that comprise mutations in position 425 and/or 441 exhibit increased efficiency in incorporating 2′-modified nucleotides into the nascent RNA chain.

In a first aspect, the present invention is thus directed to a mutein of T7 RNA polymerase, wherein the mutein comprises a mutated amino acid residue at the sequence position 425, 441 or both of the linear polypeptide sequence of T7 RNA polymerase as set forth in SEQ ID NO:1, or a functional fragment thereof, wherein the amino acid at position 425 is mutated to cysteine or tryptophane and the amino acid at position 441 is mutated to valine, leucine or tyrosine. In a preferred embodiment the mutein of T7 RNA polymerase comprises a cysteine at position 425.

In various embodiments of this first aspect of the invention, the mutein retains RNA polymerase activity. The RNA polymerase activity allows the mutein to produce an RNA molecule from a template under conditions that allow such RNA synthesis. In specific embodiments, the mutein of the invention has a polymerase activity that corresponds to 20%, 30%, 40%, 50%, 60%, 70%, 80%, 90%, 100% or more of the activity of the wildtype with respect to its capability to synthesize RNA molecules. Further, in various embodiments, the mutein is capable to use 2′-modified, in particular 2′-methoxy modified nucleotides as substrate and incorporate those into the synthesized RNA molecule. Preferably, the mutein has an increased affinity for 2′-modified nucleotides, in particular 2′-methoxy modified nucleotides compared to the wildtype.

In various embodiments, the mutein has a high degree of sequence similarity or sequence identity to the amino acid sequence of T7 RNA polymerase as set forth in SEQ ID NO: 1. This means that in certain embodiments, the mutein has at least 60, at least 70, at least 80, at least 90 or at least 95% sequence similarity to the wildtype sequence as set forth in SEQ ID NO: 1. In other embodiments, the mutein has at least 60, at least 70, at least 80, at least 90 or at least 95% sequence identity to the wildtype sequence as set forth in SEQ ID NO:1.

In some embodiments, the mutein can comprise one or more additional mutations at positions other than 425 and 441. Exemplary mutations include, but are not limited to mutations at positions E593, Y639, V685 and H784, for example E593G, Y639F, V685A, and HT784A.

In one specific embodiment, the mutein has the amino acid sequence as set forth in any one of SEQ ID Nos:2-6 or of a fragment thereof.

In another aspect, the present invention also encompasses a mutein of T7 RNA polymerase having an increased affinity for 2′-modified nucleotides, in particular 2′-methoxy modified nucleotides, compared to the wildtype, obtainable by the method of the invention.

In a further aspect, the present invention also relates to a nucleic acid molecule comprising a nucleotide sequence encoding a mutein of the invention. The nucleic acid molecule may be comprised in a vector, for example a phagemid vector.

The present invention is further directed to a host cell containing a nucleic acid molecule of the invention.

In a still further aspect, the invention features a method for producing a mutein of T7 RNA polymerase according to the invention, wherein the mutein is produced starting from the nucleic acid encoding the mutein by means of genetic engineering methods in a bacterial or eukaryotic host organism and is isolated from this host organism or its culture. The method may also be used to produce a multitude of T7 RNA polymerase muteins, thus generating a library of muteins.

The invention also encompasses the thus produced mutein library.

Also contemplated are methods for the synthesis of a partially or completely 2′-modified RNA molecule, such as a 2′-methoxy modified RNA molecule, comprising contacting a template nucleic acid with the mutein of T7 RNA polymerase according to the invention in the presence of 2′-modified ribonucleotides under conditions that allow synthesis of a 2′-modified RNA molecule by the polymerase activity of the T7 RNA polymerase mutein.

In various embodiments, the 2′-modified ribonucleotides are 2′-methoxy modified ribonucleotides. These may be selected from the group consisting of 2′-methoxy adenosine triphosphate (2′-methoxy ATP), 2′-methoxy guanosine triphosphate (2′-methoxy GTP), 2′-methoxy uracil triphosphate (2′-methoxy UTP) and 2′-methoxy cytosine triphosphate (2′-methoxy CTP) or combinations thereof. Encompassed are thus embodiments wherein only one of the four naturally occurring ribonucleotides is 2′-methoxy modified as well as embodiments wherein two, three or all four of the ribonucleotides are 2′-methoxy modified. In any of these embodiments, the reaction mixture may also comprise the respective unmodified ribonucleotides, i.e. a mixture of modified and unmodified ribonucleotides having the same base moiety, for example 2′-methoxy modified ATP as well as unmodified ATP. In certain embodiments, all substrate ribonucleotides are 2′-modified, for example 2′-methoxy ribonucleotides.

The RNA molecule synthesized by this method may be any type of RNA molecule, including but not limited to an RNA aptamer, a ribozyme, an siRNA, an miRNA or an antisense RNA. The length of the RNA molecule can vary and can for example be greater than 10, greater than 20, greater than 50, greater than 100, greater than 200, greater than 500 or even greater than 1000 nucleotides.

In a still further aspect, the invention is directed to the use of a mutein of T7 RNA polymerase according to the invention for the synthesis of a 2′-methoxy modified RNA molecule.

In another aspect, the invention also relates to RNA molecules obtainable according to the methods of producing 2′-modified RNA molecules of the invention, wherein the RNA molecule comprises one or more 2′-modified ribonucleotide units.

### BRIEF DESCRIPTION OF THE DRAWINGS

The invention will be better understood with reference to the detailed description when considered in conjunction with the non-limiting examples and the accompanying drawings.

FIG. 1 shows a representation of the nucleotide binding site of T7 RNA polymerase detailing the ribose-specific interactions between the initiating nucleotides and active site residues of T7 RNA polymerase. The Figure shows that amino acid residues R425, K441, Y639 as well as active site residues D537 and D812 are in close proximity to the ribose 2′-OH groups of both GTPs.

FIG. 2 schematically shows the combined selection/screening approach for the identification of T7 RNA polymerase variants with improved activity in presence of modified nucleotides. The left part illustrates the selection of active polymerase variants: E. coli BLR cells were co-transformed with a plasmid-encoded library of T7 RNA polymerase variants randomized at amino acid residue K441 or R425 and a compatible reporter plasmid that encodes green fluorescent protein (GFP). Transformants expressing active T7 RNA polymerase turn green due to T7-promoter-driven transcription and expression of GFP. Green colonies are transferred to a microplate for expression cultivation, lysed, and supplied with reaction buffer, primer/template as well as regular and/or modified nucleotides. The primer/template consists of a molecular beacon design {D. Summerer, A. Marx (2002) Angew. Chem. Int. Ed. Engl., 41, 3620} that was modified to encompass a T7 promoter sequence. The combination of fluorescent label (tetramethylrhodamine, shown in light grey) and quencher (dabcyl, shown in dark grey) interacts in the stem-loop state of the molecule and consequently fluorescence of the label is quenched. Upon transcription by active T7 RNA polymerase, the hairpin loop unfolds, separating the dye-quencher pair and providing for the emission of fluorescence that is detected at 590 nm (excitation at 540 nm).

FIG. 3 shows results of activity assays with lysates of E. coli BLR/pUCT7I-R441X (fluorescence reading in a microplate format). Each reaction (25 μl) contained 1 μl lysate, 0.4 μM molecular beacon (with double-stranded T7 promoter sequence), 0.2 mM each of the four NTPs (natural or modified), and 5 μg salmon sperm DNA in 1× reaction buffer. Light blue, endpoint fluorescence determination after 40 min; dark blue, initial increase of fluorescence. (A) GTP substituted by 2% O-me-GTP, (B) all natural NTPs, (C) UTP substituted by 2% O-me-UTP.

FIG. 4 shows results of activity assays with lysates of E. coli BLR/pUCT7I-R425X (fluorescence reading in a microplate format). Each reaction (25 μl) contained 1 μl lysate, 0.4 μM molecular beacon (with double-stranded T7 promoter sequence), 0.2 mM each of the four NTPs (natural or modified), and 5 μg salmon sperm DNA in 1× reaction buffer. Light blue, endpoint fluorescence determination after 40 min; dark blue, initial increase of fluorescence. (A) All natural NTPs, (B) GTP substituted by 2% O-me-GTP.

FIG. 5 shows transcription in the presence of 2% O-me-modified nucleotides. Analysis involved hybridization of the RNA transcripts with a 5′-Cy3-labeled oligonucleotide (ODN; 40 nt; SEQ ID NO:19), resolution of RNA:DNA heterodimers on native polyacrylamide gels (10%), and fluorescence scanning. The most intense band in each lane is excess labeled ODN. (A) Results of transcription by wildtype T7 RNAP in the presence of one 2% O-me-modified nucleotide as indicated. Reactions were either performed in the presence of Mg²⁺ as sole divalent ion or with addition of Mn²⁺. (B) Results of transcription by variant R425C with substitution of single nucleotides. (C and D) In vitro transcription of a 284-nt template using T7 RNAP variant R425C and substitution of rNTPs by 2% O-me-modified NTP(s). (C) Single substitution by the respective analog as indicated. (D) Multiple substitutions as indicated. M is a double-stranded fluorescent ladder (CXR, 60-400 bp; Promega). (E) Results of transcription of a 1000 nt template by variant R425C with natural NTPs (r) or substitution of single nucleotides or all nucleotides (all).

FIG. 6 shows reverse transcription of fully 2% O-me-modified RNA resulting from transcription with variant R425C and substitution of either GTP or CTP by their O-me-modified analogs. Resolution on 1% agarose (1×TAE buffer) and staining with ethidiumbromide. M, marker (Gene ruler 100 bp; Fermentas).

FIG. 7 shows the identification of constituent nucleosides resulting from complete cleavage of RNA. (A) Nucleosides released from unmodified RNA. (B) Nucleosides released from 2% O-me-modified RNA. (C) Direct comparison of RNA degradation products. The arrow indicates residual LiCl.

FIG. 8 shows functional activity of a 2% O-me-modified anti-EGFR aptamer. (A) FACS analysis of Alexa Fluor® 488-labeled anti-EGFR aptamers binding to A431 cells expressing the EGF receptor. Black, A431 control (unlabeled population); gray, A431 cells+aptamer (unlabeled population); blue, A431+aptamer bound to Alexa Fluor® 488-labeled streptavidine (labeled population). (B) Scatter plot showing all events with gate selecting intact cells. Gray, A431 (control); blue, A431+labeled aptamer. Abbreviations: FSC-A, forward light scatter; SSC-A, sideward light scatter; K, kilo (1,000). (C) All events gated as intact cells plotted for fluorescence. Gates for selection of labeled population, lower right; gates for selection of unlabeled population, upper left; gray, A431 (control); blue, A431+labeled aptamer.

### DETAILED DESCRIPTION OF THE INVENTION

The terms used herein have, unless explicitly stated otherwise, the following meanings.

“T7 RNA polymerase” or “T7 RNAP” as interchangeably used herein relates to the DNA-directed RNA polymerase of bacteriophage T7 (enterobacteria phage T7) with the UniProtKB/Swiss-Prot Accession No. P00573 (version 98 of the entry and version 2 of the sequence). The complete 883 amino acid long primary sequence is set forth in SEQ ID NO: 1. The term also includes variants and isoforms of this protein, in particular naturally occurring variants and isoforms. The polypeptide is encoded by nucleotides 3171 to 5822 of the T7 bacteriophage genome. The nucleotide sequence encoding the protein is set forth in SED ID NO:24.

The term “polymerase activity”, as used herein, relates to the enzymatic functionality of the claimed muteins and means that the mutein is capable of synthesizing an RNA molecule from substrate nucleotides that may be wildtype nucleotides and/or modified nucleotides. Polymerase activity is also considered to be present, if the mutein can use only one specific modified nucleotide as a substrate with sufficient affinity and/or only produces short molecules of only 2-10 nucleotides.

The term “variant” as used in the present invention relates to derivatives of a protein or peptide that comprise modifications of the amino acid sequence, for example by substitution, deletion, insertion or chemical modification. Preferably, such modifications do not reduce or change the functionality of the protein or peptide. Such variants include proteins, wherein one or more amino acids have been replaced by their respective D-stereoisomers or by amino acids other than the naturally occurring 20 amino acids, such as, for example, ornithine, hydroxyproline, citrulline, homoserine, hydroxylysine, norvaline. However, such substitutions may also be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

“Fragment”, as used herein, relates to an N-terminally and/or C-terminally shortened polypeptide, i.e. a polypeptide that lacks one or more of the N-terminal and/or C-terminal amino acids. Usually, the fragments are still functional, i.e. retain the biologic activity of the full length polypeptide at least to a certain extent. The fragments of the invention are preferably at least 100, more preferably at least 200, most preferably at least 300 amino acids long and retain the polymerase activity of the protein. “Biological activity” or the property of being “functional”, as used herein in relation to the muteins of the invention, may refer to an enzymatic activity of the polypeptide, the interacting potential towards other molecules and polypeptides or the cellular localization. The functional or biological activity of polypeptide variants or fragments can be 20, 30, 40, 50, 60, 70, 80, 90, 100% or more than the activity of an appropriate reference, e.g. the wildtype polypeptide. Preferably the functional or biological activity is 50% or more compared to an appropriate reference and more preferably the functional or biological activity is at least 80 or at least 90% or more compared to an appropriate reference.

“At least one”, as used herein, relates to one or more, in particular 1, 2, 3, 4, 5, 6, 7, 8, 9, 10 or more.

“Mutation” as used herein relates to a variation in the nucleotide and/or amino acid sequence of a given nucleotide sequence or protein and includes substitutions, deletions, and insertions. In one specific example, the mutation is a point mutation, i.e. the replacement of one or more nucleotides and/or amino acids in a given sequence. It is understood that if the term “mutation” is used in relation to a protein sequence, that the nucleotide sequence encoding the protein can comprise multiple mutations or modifications, including silent mutations that, for example, serve the purpose to increase expression efficiency (codon-optimization) without changing the amino acid sequence. In the present invention, the mutation is preferably the substitution of one or two amino acids by other amino acids. The term “mutein” as used herein, refers to a protein that compared to its wildtype comprises at lease one mutation in its amino acid sequence, with the mutation including the substitution, deletion and/or insertion of at least one amino acid residue.

“Sequence similarity”, as used in relation to the muteins, refers to the feature that an amino acid sequence is, with respect to its primary sequence, similar to the wildtype in that the amino acids at the positions corresponding to those of the wildtype are either identical or replaced by conservative replacements, i.e. substituted by amino acids having similar properties as the replaced amino acid. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan.

“Sequence identity”, as used in relation to the muteins, refers to the feature that an amino acid sequence is, with respect to its primary sequence, identical to the wildtype in that the amino acids at the positions corresponding to those of the wildtype are identical.

“2′-modified nucleotide”, as used herein, relates to nucleotides where the usual 2′-hydroxy group (in case of ribonucleotides) or the 2′-hydrogen (in case of desoxyribonucleotides), i.e. the hydroxy group or hydrogen at carbon 2 of the (desoxy)ribose ring, is replaced by another substituent group, such as an alkoxy group, for example methoxy (—O—CH₃) or ethoxy (—O—CH₂—CH₃). Specific examples are 2′-methoxy modified nucleotides (2′-O-methyl modified nucleotides).

“Affinity” as used herein relates to the binding characteristics, in particular the binding affinity of a protein for a given ligand that can be determined by methods known to those skilled in the art, such as spectroscopic techniques, including fluorescence spectroscopy, calorimetry, surface plasmon resonance, enzymatic assays and the like. Hence, “increased affinity” relates to a tighter binding compared to a standard, usually the wildtype, which can be determined according to any known method. It is readily apparent to the skilled person that complex formation is dependent on many factors such as concentration of the binding partners, the presence of competitors, ionic strength of the buffer system etc. Selection and enrichment is generally performed under conditions allowing the isolation of muteins having a sufficiently high dissociation constant. However, the washing and elution steps can be carried out under varying stringency. A selection with respect to the kinetic characteristics is possible as well. For example, the selection can be performed under conditions, which favor complex formation of the target with muteins that show a slow dissociation from the target, or in other words a low k_{off} rate. Alternatively, selection can be performed under conditions, which favor fast formation of the complex between the mutein and the target, or in other words a high kₒₙ rate.

The term “mutagenesis” as used herein means that the experimental conditions are chosen such that the amino acid naturally occurring at a given sequence position of T7 RNA polymerase can be substituted by at least one amino acid that is not present at this specific position in the respective natural polypeptide sequence. The term “mutagenesis” also includes the (additional) modification of the length of sequence segments by deletion or insertion of one or more amino acids. Thus, it is within the scope of the invention that, for example, one amino acid at a chosen sequence position is replaced by a stretch of three random mutations, leading to an insertion of two amino acid residues compared to the length of the respective segment of the wildtype protein. Such an insertion or deletion may be introduced independently from each other in any of the peptide segments that can be subjected to mutagenesis in the invention. The term “random mutagenesis” means that no predetermined single amino acid (mutation) is present at a certain sequence position but that at least two amino acids can be incorporated with a certain probability at a predefined sequence position during mutagenesis.

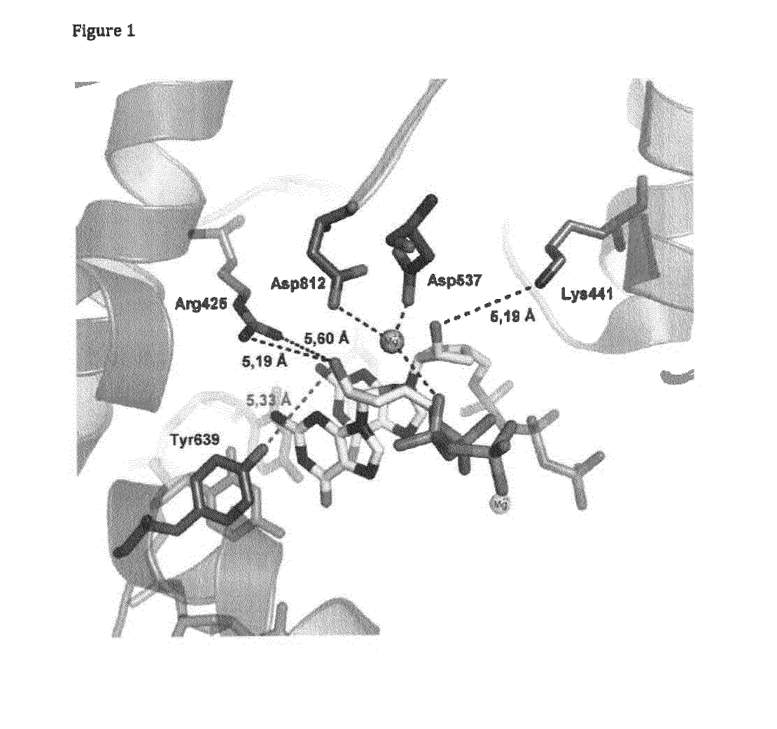

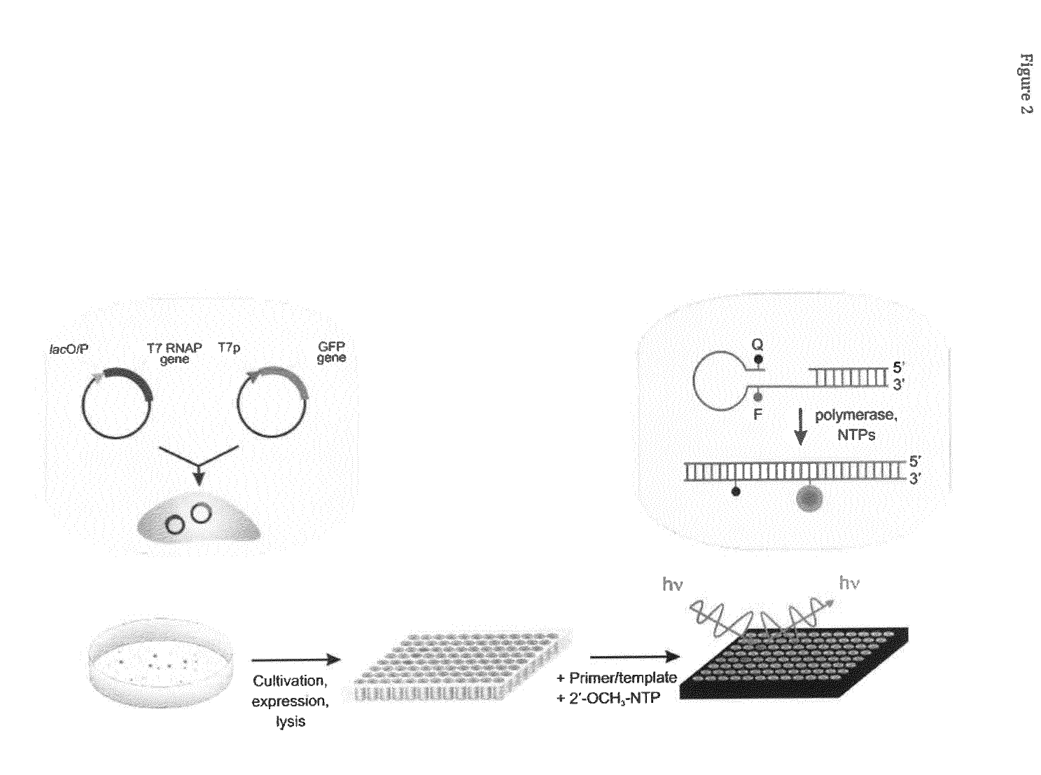

The inventors of the present invention have unexpectedly found that positions 425 and 441 of T7 RNA polymerase are critical for nucleotide recognition, binding and incorporation into the nascent RNA chain. RNA polymerases initiate RNA synthesis by recognizing a specific sequence on the DNA template, selection of the first pair of nucleoside triphosphates complementary to the template residues at positions +1 and +2, and catalyzing the formation of a phosphodiester bond to form a dinucleotide. This first catalytic stage of transcription is referred to as de novo synthesis. Bacteriophage T7 RNA polymerase (T7 RNAP) initiates transcription with a marked preference for GTP at the positions +1 and +2. The inventors identified that, in addition to K441, residues R425 and Y639 could interfere with the 2′-OH of both initiating nucleotides (see FIG. 1) and individually randomized positions 425 (wildtype: arginine) and 441 (wildtype: lysine) in order to generate mutant enzymes with improved catalytic activity in the presence of 2′-O-me-GTP.

Based on this finding, the inventors have designed muteins of T7 RNA polymerase that comprise one or two mutations in either position 425 or 441 that allows accommodating 2′-modified nucleotides, in particular increases the efficacy of binding and incorporating 2′-modified nucleotides into synthesized RNA molecules. The present invention, in a first aspect, thus relates to muteins of T7 RNA polymerase that comprise at least one mutated amino acid residue at sequence position 425 and/or 441 or a functional fragment thereof wherein the amino acid at position 425 is mutated to cysteine or tryptophane and the amino acid at position 441 is mutated to valine, leucine or tyrosine. The muteins may have an amino acid sequence that corresponds to that set forth in SEQ ID NO:1 but may comprise at least one mutated amino acid, with at least one mutation being at those sequence positions that correspond to sequence positions 425 and/or 441 of the amino acid sequence set forth in SEQ ID NO:1.

These mutations increase the enzyme's efficacy of using 2′-modified, in particular 2′-methoxy modified nucleotides as substrate and incorporating these into the synthesized RNA molecule. The increase in efficacy may be due to an increased affinity for 2′-modified nucleotides, in particular 2′-methoxy modified nucleotides compared to the wildtype.

In the muteins of the invention, the arginine residue at position 425 of the native polypeptide sequence of T7 RNA polymerase may be mutated to cysteine or tryptophane. A preferred mutein is the mutein comprising the R425C substitution. One embodiment of such a mutein has the sequence set forth in SEQ ID NO:2 or a functional fragment thereof. In case the mutein is a fragment of SEQ ID NO:2, this fragment includes the mutated amino acid position 425 and retains RNA polymerase activity. Another mutein is a mutein comprising the R425W mutation. One embodiment of such a mutein has the sequence set forth in SEQ ID NO:3 or a functional fragment thereof. In case the mutein is a fragment of SEQ ID NO:3, this fragment includes the mutated amino acid position 425 and retains RNA polymerase activity. Functional or biological active fragments of these sequences have polymerase activity.

Alternatively or additionally, the muteins may comprise a mutation of the lysine at position 441 of the linear polypeptide sequence of T7 RNA polymerase, wherein this mutation is the substitution of the native lysine by any amino acid selected from the group consisting of valine, leucine and tyrosine. In various embodiments, the mutein comprises the K441V, K441L or K441Y mutation. Exemplary embodiments of such muteins have the amino acid sequence set forth in any one of SEQ ID Nos. 4-6. Also encompassed are functional or biological active fragments of these sequences that include the mutated position and have polymerase activity.

The fact that the muteins of the invention comprise one or two mutations at positions 425 and/or 441, does not exclude that the muteins comprise further mutations at other positions of the polypeptide chain. These additional mutations may for example serve the purpose to increase stability, solubility, enzymatic activity, expression yield, specificity, selectivity and the like. Exemplary mutation positions include, but are not limited to positions 593, 639, 685 and 784 of the linear polypeptide sequence of T7 RNA polymerase as set forth in SEQ ID NO: 1. The natural amino acids at these positions may be replaced by any other amino acid. Accordingly, the invention comprises embodiments where the mutations E593G, Y639F, V685A, and/or H784A are included in the mutein.

The muteins of the invention as defined above may be generated by methods comprising mutating a nucleic acid molecule encoding a T7 RNA polymerase at one or two codons encoding any of the amino acid sequence positions 425 and 441 of the linear polypeptide sequence of T7 RNA polymerase as set forth in SEQ ID NO:1, thereby obtaining a plurality of nucleic acids encoding muteins of T7 RNA polymerase. The resulting mutant nucleic acid molecules may then be expressed in a suitable expression system to obtain the muteins. Muteins having the desired properties, i.e. have an increased affinity for 2′-modified nucleotides, for example 2′-methoxy modified nucleotides, are then enriched, for example by selection and/or isolation.

The natural coding sequence of T7 RNA polymerase, i.e. the respective gene segment of bacteriophage T7, can be used as a starting point for the mutagenesis of the amino acid positions selected in the present invention. For the mutagenesis of the recited amino acid positions, the person skilled in the art has at his disposal the various established standard methods for site-directed mutagenesis {Sambrook, J. et al. (1989) Molecular Cloning: A Laboratory Manual, 2nd Ed., Cold Spring Harbor Laboratory Press, Cold Spring Harbor, N.Y.}. A commonly used technique is the introduction of mutations by means of PCR (polymerase chain reaction) using mixtures of synthetic oligonucleotides, which bear a degenerate base composition at the desired sequence positions. For example, use of the codon NNK or NNS (wherein N=adenine, guanine or cytosine or thymine; K=guanine or thymine; S=adenine or cytosine) allows incorporation of all 20 amino acids plus the amber stop codon during mutagenesis, whereas the codon VVS limits the number of possibly incorporated amino acids to 12, since it excludes the amino acids Cys, Ile, Leu, Met, Phe, Trp, Tyr, Val from being incorporated into the selected position of the polypeptide sequence; use of the codon NMS (wherein M=adenine or cytosine), for example, restricts the number of possible amino acids to 11 at a selected sequence position since it excludes the amino acids Arg, Cys, Gly, Ile, Leu, Met, Phe, Trp, Val from being incorporated at a selected sequence position. In this respect it is noted that codons for other amino acids (than the regular 20 naturally occurring amino acids) such as selenocystein or pyrrolysine can also be incorporated into a nucleic acid of a mutein. It is also possible, as described by Wang, L. et al ((2001) Science 292, 498-500) or Wang, L., and Schultz, P. G. ((2002) Chem. Comm. 1, 1-11) to use “artificial” codons such as UAG which are usually recognized as stop codons in order to insert other unusual amino acids, for example o-methyl-L-tyrosine or p-aminophenylalanine.

The use of nucleotide building blocks with reduced base pair specificity, as for example inosine, 8-oxo-2′deoxyguanosine or 6(2-deoxy-β-D-ribofuranosyl)-3,4-dihydro-8H-pyrimidino-1,2-oxazine-7-one (Zaccolo et al. (1996) J. Mol. Biol. 255, 589-603), is another option for the introduction of mutations into a chosen sequence segment.

A further possibility is the so-called triplet-mutagenesis. This method uses mixtures of different nucleotide triplets, each of which codes for one amino acid, for incorporation into the coding sequence (Virnekäs B, Ge L, Plückthun A, Schneider KC, Wellnhofer G, Moroney SE. (1994). Nucleic Acids Res 22, 5600-5607).

One possible strategy for introducing mutations in the selected positions is based on the use of two oligonucleotides, each of which is partially derived from one of the corresponding sequence stretches wherein the amino acid position to be mutated is located. When synthesizing these oligonucleotides, a person skilled in the art can employ mixtures of nucleic acid building blocks for the synthesis of those nucleotide triplets which correspond to the amino acid positions to be mutated so that codons encoding all natural amino acids randomly arise, which at last results in the generation of a protein library.

The nucleic acid molecules defined above can be connected by ligation with missing 5′- and 3′-sequences of a nucleic acid encoding a T7 RNA polymerase polypeptide, if any, and/or the vector, and can be cloned in a known host organism. A multitude of established procedures are available for ligation and cloning (Sambrook, J. et al. (1989), supra). For example, recognition sequences for restriction endonucleases also present in the sequence of the cloning vector can be engineered into the sequence of the synthetic oligonucleotides. Thus, after amplification of the respective PCR product and enzymatic cleavage the resulting fragment can be easily cloned using the corresponding recognition sequences.

In still another aspect, the present invention also encompasses a mutein of T7 RNA polymerase having an increased affinity for 2′-modified nucleotides, in particular 2′-methoxy modified nucleotides, compared to the wildtype, obtainable by the method of the invention.

The muteins of the invention may comprise the wildtype (natural) amino acid sequence outside the mutated amino acid sequence positions. On the other hand, the muteins disclosed herein may also contain amino acid mutations outside the sequence positions subjected to mutagenesis as long as those mutations do not interfere with the binding activity and the folding of the mutein. Such mutations can be accomplished very easily on DNA level using established standard methods (Sambrook, J. et al. (1989) supra). Possible alterations of the amino acid sequence are insertions or deletions as well as amino acid substitutions. Such substitutions may be conservative, i.e. an amino acid residue is replaced with a chemically similar amino acid residue. Examples of conservative substitutions are the replacements among the members of the following groups: 1) alanine, serine, and threonine; 2) aspartic acid and glutamic acid; 3) asparagine and glutamine; 4) arginine and lysine; 5) isoleucine, leucine, methionine, and valine; and 6) phenylalanine, tyrosine, and tryptophan. One the other hand, it is also possible to introduce non-conservative alterations in the amino acid sequence.

The muteins of the invention can have a high degree of sequence similarity or sequence identity to the amino acid sequence of T7 RNA polymerase as set forth in SEQ ID NO:1 or variants and isoforms thereof, in particular naturally occurring variants and isoforms. This may mean that the mutein may have at least 60, at least 70, at least 80, at least 90 or at least 95% sequence similarity to the wildtype sequence as set forth in SEQ ID NO:1. Alternatively, the mutein may have at least 60, at least 70, at least 80, at least 90 or at least 95% sequence identity to the wildtype sequence as set forth in SEQ ID NO:1.

Possible additional modifications of the amino acid sequence include directed mutagenesis of single amino acid positions in order to simplify sub-cloning of the mutated gene or its parts by incorporating cleavage sites for certain restriction enzymes. In addition, these mutations can also be incorporated to further improve the affinity of a mutein for 2′-modified nucleotides. Furthermore, mutations can be introduced in order to modulate certain characteristics of the mutein such as to improve folding stability, protease resistance or water solubility or to reduce aggregation tendency, if necessary. It is also possible to deliberately mutate other amino acid sequence positions to cysteine in order to introduce new reactive groups, for example for the conjugation to other compounds. Exemplary mutation positions and mutations have been disclosed above.

The nucleic acid molecules of the invention comprising a nucleotide sequence encoding a mutein as described herein, may comprise additional mutations outside the indicated sequence positions of experimental mutagenesis. Such mutations are often tolerated or can even prove to be advantageous, for example if they contribute to an improved folding efficiency, serum stability, thermal stability or ligand binding affinity of the mutein.

A nucleic acid molecule disclosed in this application may be “operably linked” to a regulatory sequence (or regulatory sequences) to allow expression of this nucleic acid molecule.

A nucleic acid molecule, such as DNA, is referred to as “capable of expressing a nucleic acid molecule” or capable “to allow expression of a nucleotide sequence” if it comprises sequence elements which contain information regarding to transcriptional and/or translational regulation, and such sequences are “operably linked” to the nucleotide sequence encoding the polypeptide. An operable linkage is a linkage in which the regulatory sequence elements and the sequence to be expressed are connected in a way that enables gene expression. The precise nature of the regulatory regions necessary for gene expression may vary among species, but in general these regions comprise a promoter which, in prokaryotes, contains both the promoter per se, i.e. DNA elements directing the initiation of transcription, as well as DNA elements which, when transcribed into RNA, will signal the initiation of translation. Such regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native polypeptide to a specific compartment of a host cell.

In addition, the 3′ non-coding sequences may contain regulatory elements involved in transcriptional termination, polyadenylation or the like. If, however, these termination sequences are not satisfactory functional in a particular host cell, then they may be substituted with signals functional in that cell.

Therefore, a nucleic acid molecule of the invention can include a regulatory sequence, preferably a promoter sequence. In another preferred embodiment, a nucleic acid molecule of the invention comprises a promoter sequence and a transcriptional termination sequence. Suitable prokaryotic promoters are, for example, the tet promoter, the lacUV5 promoter or the T7 promoter. Examples of promoters useful for expression in eukaryotic cells are the SV40 promoter or the CMV promoter.

The nucleic acid molecules of the invention can also be part of a vector or any other kind of cloning vehicle, such as a plasmid, a phagemid, a phage, a baculovirus, a cosmid or an artificial chromosome.

Such cloning vehicles can include, aside from the regulatory sequences described above and a nucleic acid sequence encoding a mutein of the invention, replication and control sequences derived from a species compatible with the host cell that is used for expression as well as selection markers conferring a selectable phenotype on transformed or transfected cells. Large numbers of suitable cloning vectors are known in the art, and are commercially available.

The DNA molecule encoding muteins of the invention, and in particular a cloning vector containing the coding sequence of such a mutein can be transformed into a host cell capable of expressing the gene. Transformation can be performed using standard techniques (Sambrook, J. et al. (1989), supra). Thus, the invention is also directed to a host cell containing a nucleic acid molecule as disclosed herein.

The transformed host cells are cultured under conditions suitable for expression of the nucleotide sequence encoding a fusion protein of the invention. Suitable host cells can be prokaryotic, such as Escherichia coli (E. coli) or Bacillus subtilis cells.

To produce a mutein of the invention, a fragment of the mutein, or a fusion protein of the mutein and another polypeptide, the nucleic acid coding for the mutein can be genetically engineered for expression in a suitable system. The method can be carried out in vivo, the mutein can for example be produced in a bacterial or eukaryotic host organism and then isolated from this host organism or its culture. It is also possible to produce a protein in vitro, for example by use of an in vitro translation system.

When producing the mutein in vivo a nucleic acid encoding a mutein of the invention is introduced into a suitable bacterial or eukaryotic host organism by means of recombinant DNA technology (as already outlined above). For this purpose, the host cell is first transformed with a cloning vector comprising a nucleic acid molecule encoding a mutein of the invention using established standard methods (Sambrook, J. et al. (1989), supra). The host cell is then cultured under conditions, which allow expression of the heterologous DNA and thus the synthesis of the corresponding polypeptide. Subsequently, the polypeptide is recovered either from the cell or from the cultivation medium.

However, a mutein of the invention may not necessarily be generated or produced only by use of genetic engineering. Rather, a mutein can also be obtained by chemical synthesis such as Merrifield solid phase polypeptide synthesis or by in vitro transcription and translation. It is for example possible that promising mutations are identified using molecular modeling. Subsequently, the wanted (designed) polypeptide may be in vitro synthesized and then the binding activity for a given target may be investigated. Methods for the solid phase and/or solution phase synthesis of proteins are well known in the art.

In another embodiment, the muteins of the invention may be produced by in vitro transcription/translation employing well-established methods known to those skilled in the art.

The above production methods may be used to generate a library of T7 RNA polymerase mutants. This library may then be subject to screening and selection procedures, as well as further rounds of mutagenesis, for example random mutagenesis, at additional positions.

The invention also covers a thus produced library of T7 RNA polymerase muteins.

Using the T7 RNA polymerase muteins of the invention, partially or completely 2′-modified RNA molecules, such as a 2′-methoxy modified RNA molecules, may be synthesized. For such a method to synthesize a partially or completely 2′-modified RNA molecule, a template nucleic acid is contacted with the mutein of T7 RNA polymerase in the presence of 2′-modified ribonucleotides under conditions that allow synthesis of a 2′-modified RNA molecule by the polymerase activity of the T7 RNA polymerase mutein.

The 2′-modified ribonucleotides may be 2′-methoxy modified ribonucleotides, such as 2′-methoxy adenosine triphosphate (2′-methoxy ATP), 2′-methoxy guanosine triphosphate (2′-methoxy GTP), 2′-methoxy uracil triphosphate (2′-methoxy UTP), 2′-methoxy cytosine triphosphate (2′-methoxy CTP) and/or combinations thereof. In some embodiments of these methods, only one of the four naturally occurring ribonucleotides may be 2′-methoxy modified. In other embodiments, two, three or all four of the ribonucleotides are 2′-methoxy modified. In any of these embodiments, the reaction mixture may also comprise the respective unmodified ribonucleotides, i.e. a mixture of modified and unmodified ribonucleotides having the same base moiety, for example 2′-methoxy modified ATP as well as unmodified ATP.

The RNA molecule synthesized by this method may be any type of RNA molecule, including but not limited to an RNA aptamer, a ribozyme, a siRNA, a miRNA or an antisense RNA. The length of the RNA molecule can vary and can for example be greater than 10, greater than 20, greater than 50, greater than 100, greater than 200, greater than 500 or even greater than 1000 nucleotides.

The muteins of T7 RNA polymerase according to the invention may thus also be used for the synthesis of a 2′-methoxy modified RNA molecule.

In one aspect, the present invention also encompasses the RNA molecules obtainable according to the methods of the invention. These RNA molecules may be as defined above. In some embodiments, such modified RNA molecules comprise one or more 2′-modified ribonucleotides units. In such an RNA molecule all nucleotides of one type, e.g. all G or all C nucleotides, may be modified, or all nucleotides or two, three or all four types may be modified. Also encompassed are embodiments where one or more but not all nucleotides of one, two, three or four types of nucleotides are modified. The thus produced RNA molecules may be of any length, but are preferably at least 50, at least 70, at least 100, at least 150, at least 200, or at least 250 nucleotides in length.

The modified RNA molecules of the invention or produced according to the methods and uses of the invention may be used for therapeutic, diagnostic or biotechnological purposes, for example as RNA interference agents, such as siRNA, miRNA, antisense RNA, ribozymes, as probes, primers, or as research tools. Other applications of modified RNA molecules are known to those skilled in the art.

The present invention is further illustrated by the following examples. However, it should be understood, that the invention is not limited to the exemplified embodiments.

### EXAMPLES

### Materials and Methods

### A. Bacterial Strains and Plasmids

Escherichia coli XL1-Blue as well as XL1-Blue MR (Stratagene, Amsterdam, The Netherlands) were used in cloning experiments, while BL21 (Stratagene) and BLR (Novagen/Merck Chemicals Ltd., Nottingham, UK) were employed for selection/screening and protein expression.

Plasmid for Mutagenesis and Soluble Expression of T7 RNA Polymerase (T7 RNAP).

A 2.8-kb fragment coding for T7 RNAP was PCR-amplified starting from plasmid pAR1219 {P. Davanloo et al. (1984) Proc. Natl, Acad. Sci. USA, 81, 2035} using the primer pair 5′-AAAAAAAAAAAAGTCGACTTACGCGAACGCGAAGTC-3′ (SEQ ID NO:3) and 5′-AAAAAAAAAAAAAAGCTTACTGAACACGATTAACATC-3′ (SEQ ID NO:4) and Pfu DNA polymerase (Fermentas, St. Leon-Rot, Germany), digested with HindIII and SalI, and ligated with linear pUC19 {C. Yanisch-Perron, J. Vieira, J. Messing (1985) Gene, 33, 103} to yield plasmid pUCT7.

For cis-regulation of the lac promoter and inducible expression of T7 RNAP, the lacIq-coding fragment was inserted into pUCT7. Therefore, the plasmid was digested with AatII and ligated with a synthetic double-stranded fragment 5′-AAAAGACGTCAAACTCGAGAAAGACGTCAAAA-3 ‘-TTTTGACGTCTTTCTCGAGTTTGACGTCTTTT-3’ (SEQ ID NO:9/SEQ ID NO:10) that was digested accordingly. The product plasmid was digested with XhoI and SalI and ligated with the fragment excised from pREP4 (Qiagen, Hilden, Germany) by SalI. Plasmid pUCT7I that resulted from this reaction allowed for IPTG-inducible expression of soluble T7 RNAP in cultures of E. coli BLR. As a control, plasmid pUC19I was constructed according to pUCT7I by inserting the lacIq-coding fragment into pUC19.

Reporter Plasmid.

The approach for screening of active T7 RNAP variants was based on co-transformation of E. coli BLR with pUCT7I and reporter plasmid. Since BLR cells require cultivation in the presence of tetracycline, the inactive chloramphenicol resistance of the original reporter plasmid, pAlterGFP (tet^{res}, cam^{sens} {S. Brakmann, S. Grzeszik (2001) ChemBioChem, 2, 212}), was reconstructed by substitution of the mutant gene fragment with the original one from pACYC184 {A. C. Y. Chang, S. N. Cohen, J. Bacteria 1978, 134, 1141}. pACYC184 was digested with DraI releasing a 340-bp-fragment that was ligated with DraI-linearized pAlterGFP. The resulting plasmid, pAlterGC, contained the restored gene coding for chloramphenicol acetyl transferase and rendered BLR cells resistant to 34 μg/ml chloramphenicol.

### Example 1

### Generation of 7 RNAP Mutant Libraries by Saturation Mutagenesis

Site-specific saturation mutagenesis was performed using QuikChange® site-directed mutagenesis kit (Stratagene) according to the manufacturer's protocols. Mutagenesis started from plasmid pUCT7I using the primers given in Table 1. The resulting plasmid libraries were used to transform XL1-Blue cells that were plated on LB media (10 g/L tryptone, 5 g/L yeast extract, 10 g/L NaCl, and 15 g/L agar) containing ampicillin (100 μg/mL) and cultivated over night at 37° C. Colonies were pooled and directly submitted to plasmid preparation using the QIAprep Spin Miniprep Kit (Qiagen) yielding the mutant libraries pUCT7-R425X or pUCT7-K441X, respectively.

TABLE 1

| Variant | Primer sequence |
|---|---|
| R425X | Forward: 5′-CAACATG |
| | GACTGGCGCGGTNNBGTTT |
| | ACGCTGTGTCAATG-3′ |
| | (SEQ ID NO: 11) |
| | Reverse: 5′-CATTGAC |
| | ACAGCGTAAACVNNACCGC |
| | GCCAGTCCATGTTG-3′ |
| | (SEQ ID NO: 12) |
| K441X | Forward: 5′-GCAAGGT |
| | AACGATATGACCNNSGGAC |
| | TGCTTACGCTGGC-3′ |
| | (SEQ ID NO: 13) |
| | Reverse 5′-CGCCAGCG |
| | TAAGCAGTCCSNNGGTCAT |
| | ATCGTTACCTTGC-3′ |
| | (SEQ ID NO: 14) |

(N = A, G, C, T; B = G, T, C; V = G, A, C; S = G, C)

### Example 2

### Selection of Active T7 RNAP Variants

Competent BLR/pAlterGC cells were transformed with one of the mutant libraries (pUCT7-R425X, or pUCT7-K441X), subsequently plated on LB media containing ampicillin (100 μg/mL) and chloramphenicol (34 μg/mL) and cultivated at 37° C. (24 h), followed by incubation at 20° C. (12-48 h). Transformants expressing active variants of T7 RNAP appeared as green fluorescent colonies after this period of time while transformants expressing inactive T7 RNAP remained white (FIG. 2). Green colonies were selected and used for activity-based screening. This selection step yielded approx. 10% (K441X) or 5% (R425X) generally active variants (transformation efficiencies: 10⁸ cfu/μg DNA).

### Example 3

### Expression of T7 RNAP and Preparation of Cell Lysates in Microplates

Expression.

Transformants expressing active T7 RNAP variants were cultivated in a 96-well-microplate format. Fresh, green colonies of BLR/pAlterGC/pUCT7I (or, variant) were used to inoculate 1 mL of YT medium (8 g/L tryptone, 5 g/L yeast extract, 5 g/L NaCl, pH 7.0) supplemented with appropriate antibiotics. After sealing the plates with Air Pore Tape sheets (Qiagen), the cultures were shaken for 19 h at 37° C. and 220 rpm. These overnight cultures were diluted 50-fold into fresh medium (1.5 mL/well) and grown until the optical density at 600 nm reached 0.7-0.9 (spot-checked). Protein expression was induced by the addition of IPTG (final concentration: 1 mM), and incubation was continued over night. Cells were harvested by centrifugation (3,700 rpm, 5 min, 4° C.; microplate buckets; centrifuge 5804R, Eppendorf, Hamburg, Germany), freed from supernatant and stored at −20° C. until used.

Lysis.

Cell pellets were resuspended by shaking (10 min, 550 rpm, 4° C.) in 50 μL lysis buffer 1 (50 mM Tris-HCl, pH8.0, 15 mM EDTA) and treated with 10 μL 200 mM NaOH and shaking (10 min, 550 rpm, 4° C.). After neutralization with 5 μL of buffer N (1 M Tris-HCl, pH 8.0, 4 M NaCl), lysis was continued with the addition of 10 μL of lysozyme buffer (50 g/L lysozyme, 25 mM DTT) and incubation for 1 h at 550 rpm and 4° C. Cell debris (“pellet”) was separated by centrifugation (3,700 rpm, 5 min, 4° C.) leaving soluble protein in the supernatant (“lysate”) that was transferred to fresh microplates and stored at 4° C.

### Example 4

### Expression and Purification of T7 RNAP Variants

Expression.

In order to obtain sufficient amounts of soluble protein for the purification of T7 RNAP variants, expression cultivation was performed on the 100-mL-scale under microaerobic conditions (50-mL tubes with 30-40-mL portions of LB medium containing appropriate antibiotics). After inoculation with a fresh overnight culture (1:50), cultures were grown at 37° C. and 160 rpm until the optical density at 600 nm reached 0.4-0.7. Protein expression was induced with 1 mM IPTG, and incubation was continued overnight at 37° C. and 160 rpm. Cells were harvested by centrifugation (5,000 rpm, 15 min, 4° C.) and stored at −20° C. until used.

Protein Purification.

The protocol was based on an established method {J. Grodberg, J. J. Dunn (1988) J. Bacteriol., T70, 1245} with modifications as described by Zawadzki {V. Zawadzki, H. J. Gross (1991) Nucl. Acids Res., 19, 1948}. Proteins showed purities of ≧95% and typical yields of 3-5 mg per liter of culture.

### Example 5

### Assay of T7 RNA Polymerase Activity

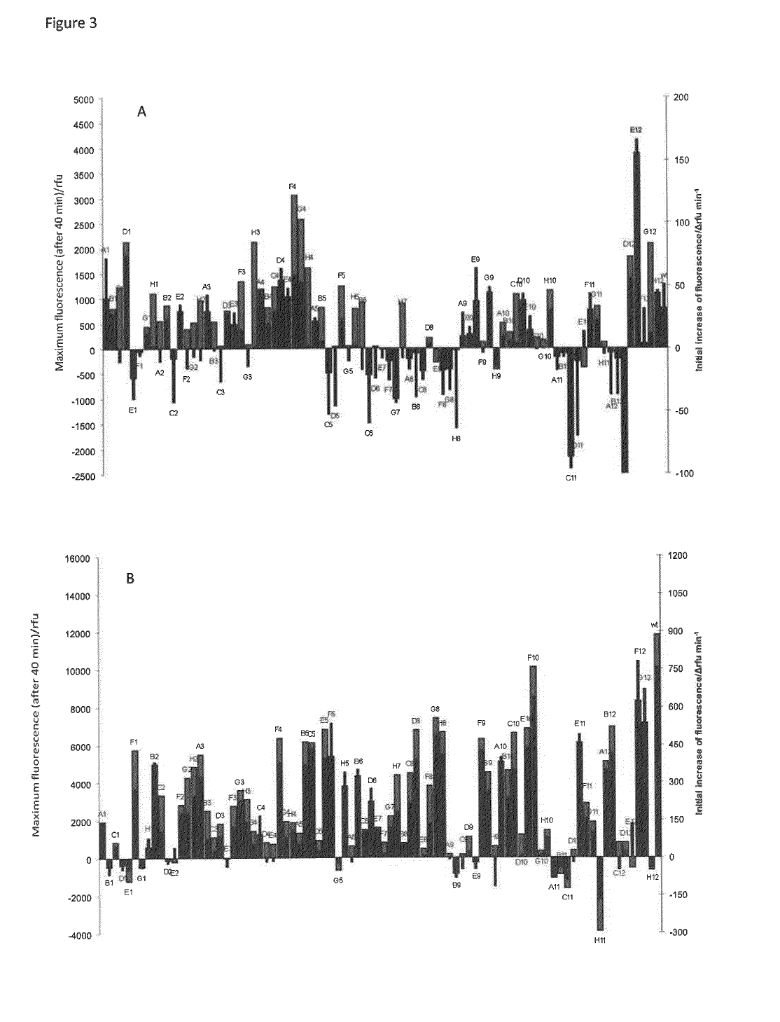

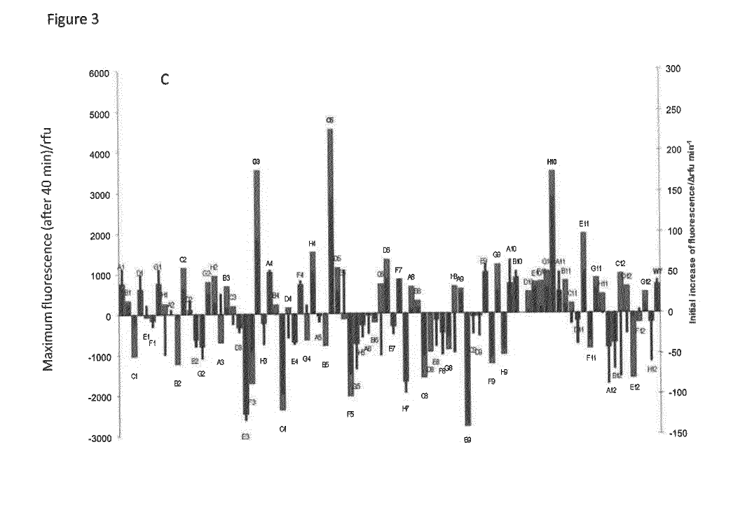

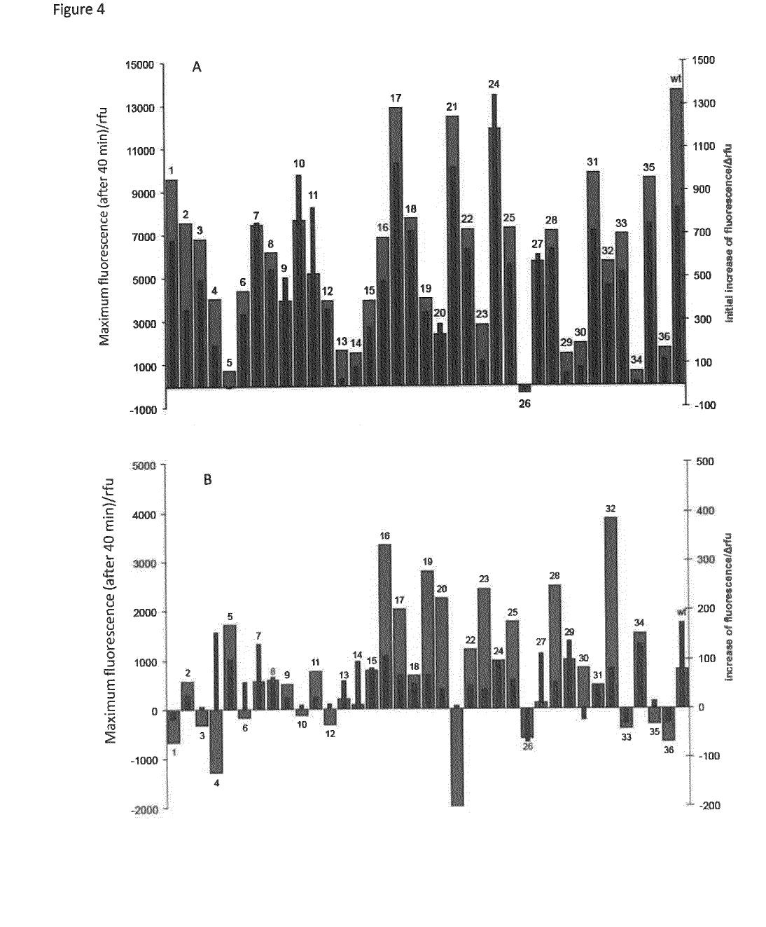

Enzymatic activity of cloned T7 RNAP or its variants was determined using a fluorescence-based assay that detected the DNA-dependant RNA polymerase activity with a molecular beacon-based primer-template {D. Summerer, A. Marx (2002) Angew. Chem. Int. Ed. Engl., 41, 3620}. The beacon 5′-GCGAGAXCCAAAAAAAAAAACCAYCTCGCCGAATTCGCCCTATAGTGAGTC GTATTA-3′ (SEQ ID NO:11) with X=Dabcyl-dT (Dabcyl, 4-(dimethylamino-azo)benzene-4-carboxylic acid) and Y=TAM′-dT (0.4 μM; IBA, Göttingen, Germany) was hybridized to the T7 promoter-containing oligonucleotide 5′-TAATACGACTCACTATA-3′ (SEQ ID NO:12) (0.44 μM) in 1× reaction buffer (40 mM Tris-HCl (pH 8,0), 30 mM MgCl₂, 10 mM DTT, 6 mM spermidine), supplied with NTPs (200 μM each) as well as 2′-O-me NTPs (200 μM with lysates, 2 mM with purified T7 RNAP) and, in the case of lysates, salmon sperm DNA (5 μg/μL lysate). The reaction was started with purified T7 RNAP (0.4 μM) or lysates (1 μL) of cultures expressing active T7 RNAP variants and allowed to proceed at 37° C. Fluorescence intensities were monitored using a microplate reader (Synergy HT; Biotek, Bad Friedrichshall, Germany) with excitation at 540 nm and emission reading at 590 nm. Control experiments were performed in analogous reaction setups but without NTPs. The assay protocol is schematically depicted in FIG. 2. FIGS. 3 and 4 show the results of activity assays with lysates of E. coli BLR/pUCT7I-R425X or E. coli BLR/pUCT7I-R441X (fluorescence reading in a microplate format). Each reaction (25 μl) contained 1 μl lysate, 0.4 μM molecular beacon (with double-stranded T7 promoter sequence), 0.2 mM each of the four NTPs (natural or modified), and 5 μg salmon sperm DNA in 1× reaction buffer. Light grey bars show the endpoint fluorescence determination after 40 min; dark grey bars show the initial increase of fluorescence. FIG. 3 shows results of activity assays for the T7 RNAP library K441X (A) with 2′-OMe-GTP instead of GTP, (B) with NTPs and (C) with 2′-OMe-UTP. FIG. 4 shows results of activity assays for the T7 RNAP library R425X (A) with 2′-OMe-GTP instead of GTP and (B) with NTPs. The results show that a number of the T7 RNAP variants exhibit polymerase activity for natural nucleotides as well as 2′-OMe-GTP and 2′-OMe-UTP.

### Example 6

### In Vitro Transcription by T7 RNA Polymerase and Variants

Template.

The DNA template for in vitro transcription was generated by PCR using pAlterGC and primers 5′-AGGCCTCTAGACTGCAGC-3′ (SEQ ID NO:17) and 5′-CGTAACTTGTGGTATCGTG-3′ (SEQ ID NO:18) for the amplification of a 536 bp fragment. The PCR product was purified by phenol/chloroform extraction and precipitation with ethanol and used without further treatment.

Transcription and Analysis of RNA Transcripts.

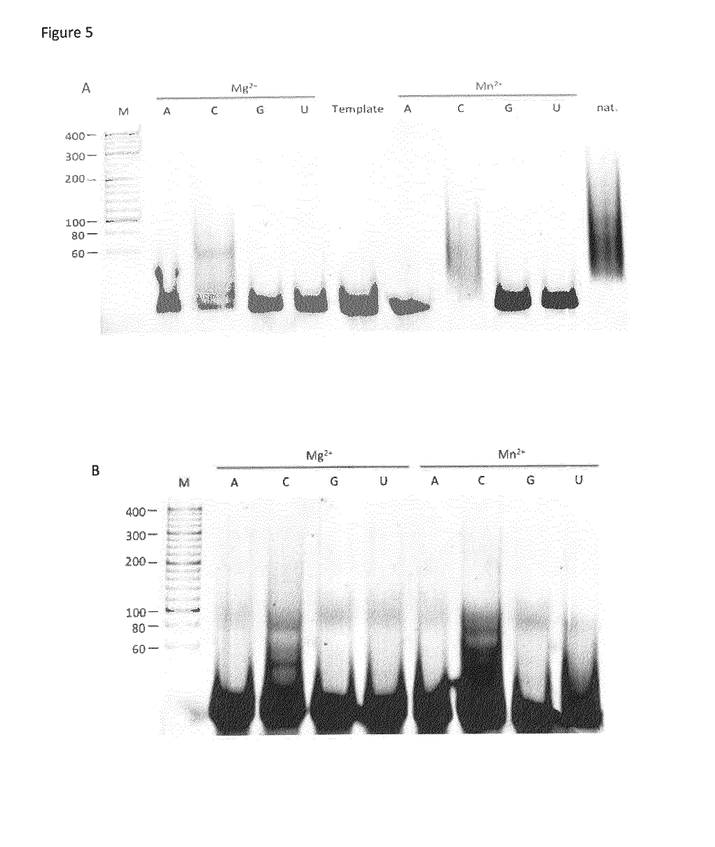

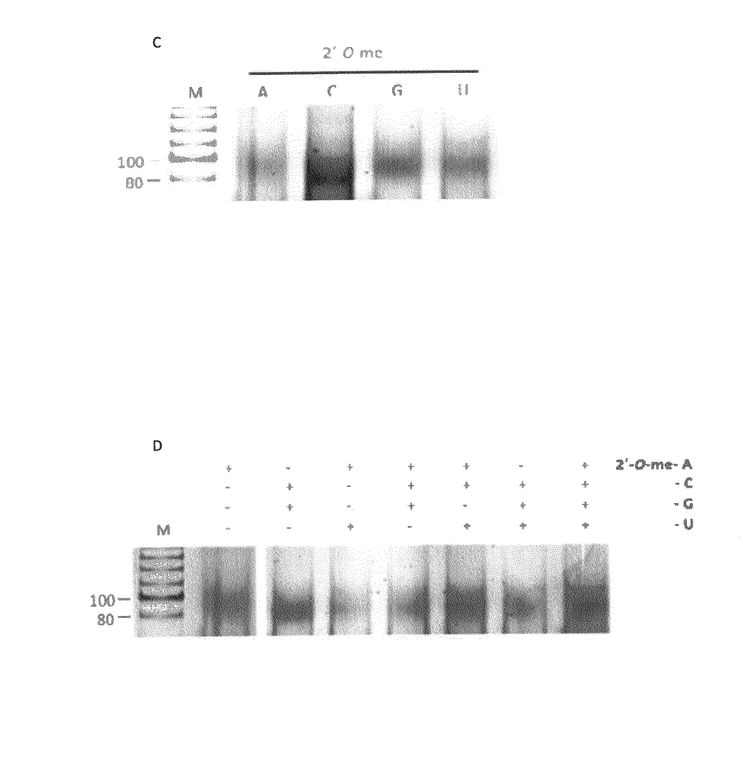

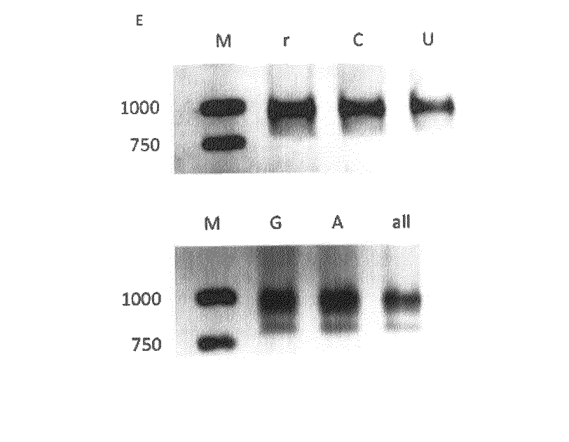

The product DNA contained a 17-bp T7 promoter sequence at position 236 and served as a template for 284-nt transcripts. DNA (200 nM) and T7 RNAP or variants (200 nM) were allowed to react in reaction buffer (200 mM HEPES, pH 7.5, 40 mM DTT, 2 mM spermidine, 8 mM MgCl₂ and, optionally, 1.5 mM MnCl₂; {P. Burmeister et al. (2006) Oligonucleotides, 16, 337}) supplemented with 3.75 mM NTPs (Fermentas) and 2′-O-me-NTPs (Trilink Biotechnologies, San Diego, Calif., USA), 10% PEG-8000, 0.01% Triton X-100 during 19 h at 37° C. The product mixture was hybridized to oligodeoxynucleotide (ODN) 5′-Cy3-CTTCTCCTTTGCTAGCCATATGTATATCTCCTTCTTAAAG-3′ (SEQ ID NO:19) at a ratio of 3:1 (ODN:template), separated by native gel electrophoresis (10% polyacrylamide) at 4° C., and visualized using a fluorescence scanner (Typhoon Trio Plus Variable, GE Healthcare, München, Germany). Image analysis was performed using ImageJ software {W. Rasband (2004) http://rsb.info.nih.gov/}. The results of this assay are shown in FIG. 5. The most intense band in each lane is excess labeled ODN. (A) Results of transcription by wildtype T7 RNAP in the presence of one 2′-O-me-modified nucleotide as indicated. Reactions were either performed in the presence of Mg²⁺ as sole divalent ion or with addition of Mn²⁺. (B) Results of transcription by variant R425C with substitution of single nucleotides. (C and D) Transcription by R425C in the presence of combinations of 2′-O-me-modified nucleotides. The results demonstrate that, in contrast to the wildtype, the variant R425C can use all 2′-O-me-modified nucleotides as substrate either alone or in combination. Further, a 284 nt template was used in in vitro transcription experiments. Run-off transcriptions were performed with linear template pAlterGFP. DNA (1.5 nM) and T7 RNAP variant R425C (150 nM) were allowed to react in 2× transcription buffer (80 mM Tris-HCl, pH 8.9, 16 mM MgCl₂, 20 mM NaCl, 4 mM spermidine, 60 mM DTT) supplemented with 2 mM NTPs or 2′-OMe-NTPs during 3 h at 37° C. After removal of template DNA by digestion with DpnI nuclease (1 u), the product RNA was analyzed using agarose gel electrophoresis (1% agarose in 1×TAE buffer containing 0.1% NaOCl) and staining with ethidium bromide. (E) Results of transcription of the 1000 nt template by variant R425C with substitution of single or all nucleotides. The results demonstrate the capability of variant R425C to use all 2′-O-me-modified nucleotides as substrate.

Purification.

Product RNA was purified using solid phase extraction (High Pure RNA Isolation Kit; Roche Diagnostics, Mannheim, Germany) according to the manufacturer's instructions and dissolved in H₂O.

### Example 7

### RNA Sequence Analysis

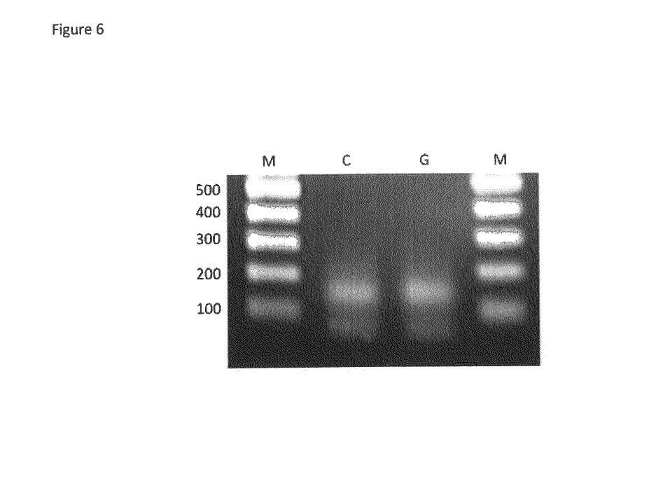

In order to allow for reverse transcription of transcripts with varying lengths, the 3′ A termini were ligated to oligonucleotide 5′-pU-ATACTCATGGTCATAGCTGTT (SEQ ID NO:20). For that equimolar amounts of RNA and oligonucleotide were dissolved in T4 RNA ligase buffer (5 mM Tris-HCl, pH 7.8, 1 mM MgCl₂, 0.1 mM ATP, 1 mM dithiothreitol; New England Biolabs, Frankfurt am Main, Germany), supplemented with 1 mM hexammine cobalt chloride, 12.5% PEG 8000 and 0.2 mg/ml BSA as well as T4 RNA ligase (10 U/μg RNA; New England Biolabs) and reacted over night at 16° C. Reverse transcription was achieved by mixing 40 μL of the ligation reaction (approx. 250-500 ng RNA) with 12 μL 5× first strand buffer (250 mM Tris-HCl (pH 8.3 at room temperature), 375 mM KCl, 15 mM MgCl₂; Gibco), addition of primer 5′-AACAGCTATGACCATGAGT-3′ (SEQ ID NO:21) as well as 0.5 μL Superscript II reverse transcriptase (100 U; Gibco) and incubation for 1 h at 42° C. The results of the RT reaction of 2′-O-me-modified RNA resulting from transcription with variant R425C and substitution of either the first GTP (G) or CTP (C) by their O-me-modified analogs with resolution on 1% agarose (1×TAE buffer) and staining with ethidiumbromide is shown in FIG. 6.

Second strand synthesis and amplification were performed by addition of 60 μL of the reverse transcription reaction with a PCR mix containing additional first strand primer, second strand primer 5′-CTTTAAGAAGGAGATGGATCCGTGGCTAGCAAAGGAGAAG-3′ (SEQ ID NO:22), 250 μM each of four dNTPs, and High Fidelity PCR Enzyme Mix (1.5 U; Fermentas) in 1× reaction buffer High Fidelity PCR Buffer, Fermentas; 1.5 mM Mg²⁺) during 25 cycles of PCR. The product was directly ligated with linear TA vector pCR2.1 (Invitrogen) according to the manufacturers's protocol and used to transform XL1-Blue cells. Plasmids were isolated from randomly chosen transformants and sequenced using primer 5′-CAGGAAACAGCTATGAC-3′ (SEQ ID NO:23). The mutation rate was determined as the number of mutations divided by the number of nucleotides sequenced.

### Example 8

### HPLC Analysis

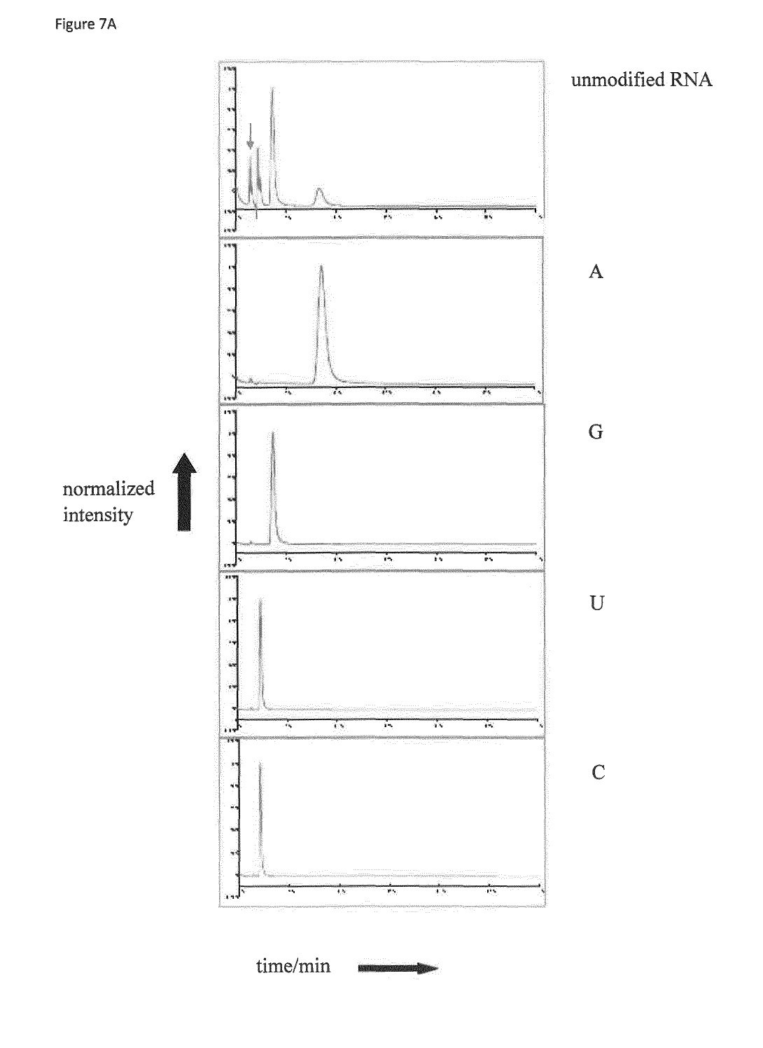

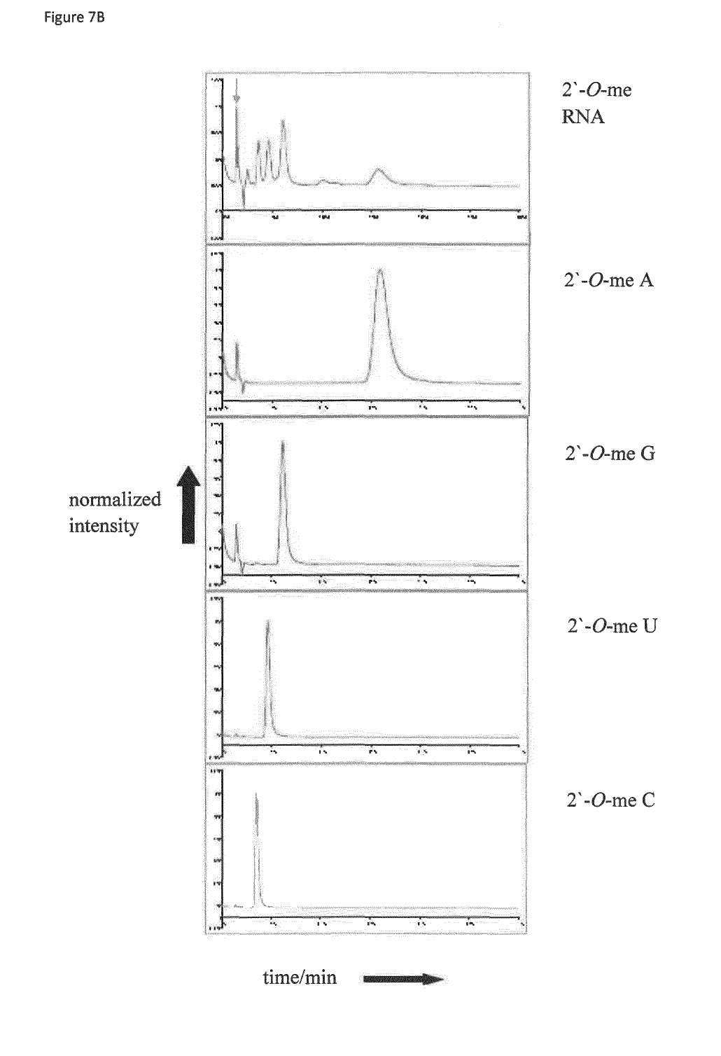

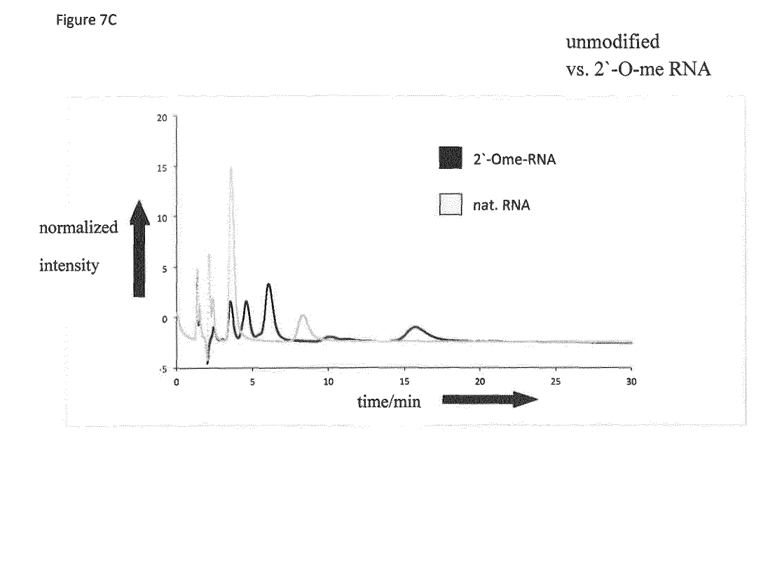

Samples for HPLC were prepared by complete cleavage of 1000-nt-transcripts in an enzymatic three-step-process as described by Helm et al. {Y. Motorin et al. (2010) Nucl. Acids Res., 39, 1943}. According to this procedure, RNA is digested with nuclease P1, further degraded by endonucleolytic cleavage with phosphodiesterase and subsequently, dephosphorylated with alkaline phosphatase. RNA (modified or unmodified) produced by in vitro transcription was freed of template DNA, collected by precipitation with LiCl/i-PrOH and dissolved in water. Endonucleolytic digestion of 3 μg RNA was achieved with 4 u Nuclease P1 (from Penicillium citrinum; Sigma-Aldrich, Taufkirchen, Germany) in 60 μl reaction buffer (10 mM NH4Ac, pH 5.4, 0.1 mM ZnCl2) during 1.5 h at 50° C. The mixture was then supplied with 0.005 u snake venom phosphodiesterase (from Crotalus adamanteus; Sigma-Aldrich) and incubated for 2 h at 37° C. In the final step, calf intestinal alkaline phosphatase (CIAP; 3 u; Roche, Mannheim, Germany) was added and allowed to react during further 2.5 h at 37° C. After separation of enzymes and other high molecular weight compounds using Vivaspin 500 columns (MWCO 5000; Sartorius, Göttingen, Germany), samples were directly applied to a Nucleodur C18 Gravity column (5 μm particle size, 110 Å pore size, 100 mm length; Macherey und Nagel, Düren, Germany), pre-equilibrated with mobile phase (85 mM NH4Ac, pH 4.6, 3% Acetonitrile) and resolved at a flowrate of 1 ml min⁻¹ (FIG. 7). Analysis was achieved with an Agilent 1100 system equipped with diode array detector monitoring at 254 nm. For peak assignment, individual nucleotides (100 nmole) were submitted to dephosphorylation using CIAP (2 u) in 20 μl of 1× CIAP buffer (Roche) during 1 h at 37° C. The reaction mixture was freed from enzyme with Vivaspin 500 columns as described above, and the resulting filtrates were directly used for HPLC analysis. The identity of all nucleosides was cross-checked with ESI-MS spectrometry (data not shown). Separation of the resulting nucleosides by reversed-phase HPLC and analysis by UV detection showed a set of peaks that could be assigned to the four 2′-O-me-modified nucleosides and that were clearly distinguishable from the set of peaks associated with natural nucleosides (FIG. 7A-C). Thus, the data confirmed that the product synthesized by T7 RNAP R425C from 2′-O-me-modified nucleotides is a fully modified RNA.

### Example 9

### Generation and Assay of a Functional Anti-EGFR Aptamer

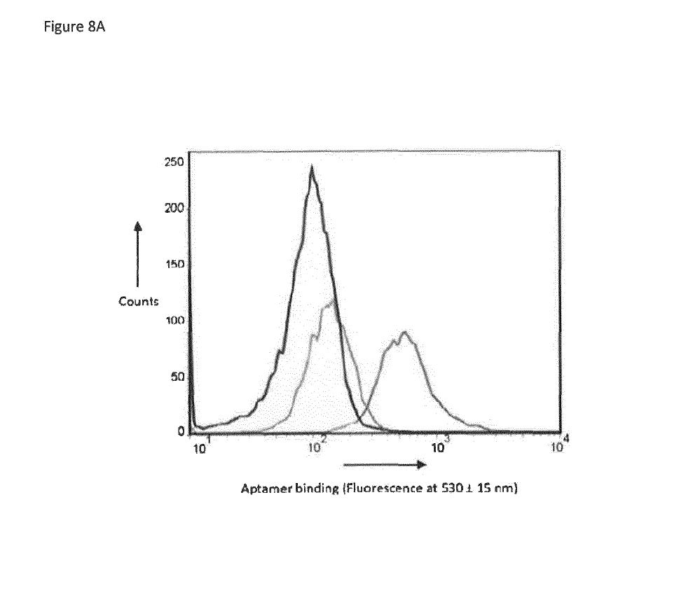

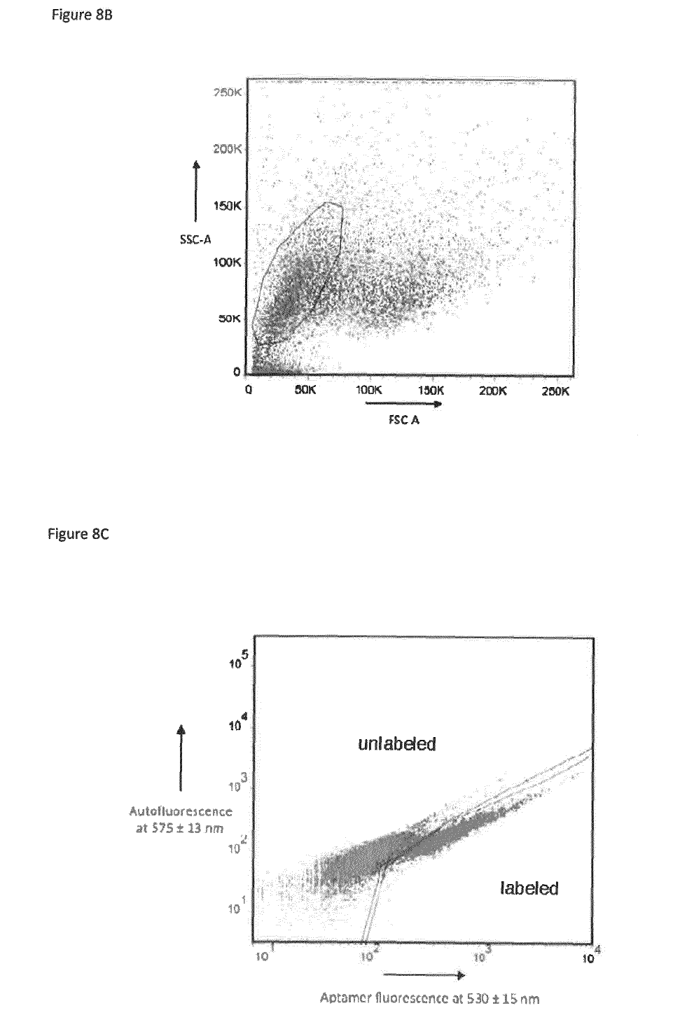

Anti-EGFR aptamer RNA based on results previously published by Li et al. {N. Li et al. (2011) PLoS ONE, 6, e200299} was synthesized starting from a template DNA, 5′-GATAATACGACTCACTATAGGCGCTCCGACCITAGTCTCTGTGCCGCTATAA TGCACGGATTTAATCGCCGTAGAAAAGCATGTCAAAGCCGGAACCGTGTAGC ACAGCAGA-3′, that consisted of a 93-nt aptamer-coding sequence flanked 5′ terminally by the T7 RNA polymerase promoter (17+2 nt; underlined) and 3′ terminally by an oligonucleotide binding site (20 nt; italics). Transcripts were hybridized to the respective, biotinylated DNA oligonucleotide and then reacted at a ratio of 2:1 with Alexa Fluor® 488 Streptavidin conjugate (Invitrogen, Darmstadt, Germany). A431 cells which express abnormally high levels of epidermal growth factor receptor (EGFR) were purchased from ATCC (American type Culture Collection, Manassas, Va., USA) and used for FACS-based analysis of aptamer binding. These cells were grown in DMEM with 10% FCS (both PAN Biotech, Aidenbach, Germany) at 37° C. and 5% CO2 to 70%, washed with DPBS (PAN Biotech), trypsinized with 0.05% trypsin-EDTA (PAN Biotech) and counted. Alexa Fluor® 488-labeled RNA (50 nM) was incubated with 0.5 million cells in 200 μl transcription buffer during 30 min at 25° C. As a negative control, an equal amount of cells was incubated in transcription buffer without aptamer. Next, cells were washed three times with binding buffer (DPBS+5 mM MgCl₂), resuspended in 300 μl binding buffer and analyzed using a BD™ LSR II (BD Biosciences). For each binding reaction, 10,000 events from intact cells were collected and analyzed using FACSDiva Version 6.1.1 software. For plotting, data was imported to FowJo Version 7.6.5 software. Fluorescence signals were plotted against counts, and intact cells were identified by using a plot of FSC-A (forward scatter) against SSC-A (sideward scatter; FIG. 8). Using this procedure, it was possible to detect the presence of fluorescently labeled cells and thus, the binding of the fully modified apatamer to the EGF receptor was verified (FIG. 8 A-C).

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Other embodiments are within the following claims. In addition, where features or aspects of the invention are described in terms of Markush groups, those skilled in the art will recognize that the invention is also thereby described in terms of any individual member or subgroup of members of the Markush group.

One skilled in the art would readily appreciate that the present invention is well adapted to carry out the objects and obtain the ends and advantages mentioned, as well as those inherent therein. Further, it will be readily apparent to one skilled in the art that varying substitutions and modifications may be made to the invention disclosed herein without departing from the scope and spirit of the invention. The compositions, methods, procedures, treatments, molecules and specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims. The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The content of all documents and patent documents cited herein is incorporated by reference in their entirety.

1-11. (canceled)

12. A T7 RNA polymerase mutein, comprising a T7 RNA polymerase or a functional fragment thereof having at least one amino acid sequence mutation at a mutated amino acid sequence position compared to a wildtype amino acid at a corresponding wildtype amino acid sequence position in a wildtype T7 RNA polymerase, wherein either or both of:
(i) the mutated amino acid sequence position corresponds to amino acid sequence position 425 of SEQ ID NO:1 and the amino acid sequence mutation is selected from cysteine and tryptophan, and
(ii) the mutated amino acid sequence position corresponds to amino acid sequence position 441 of SEQ ID NO:1 and the amino acid sequence mutation is selected from valine, leucine and tyrosine.

13. The T7 RNA polymerase mutein of claim 12, wherein either or both of:
the mutein has RNA polymerase activity; and
the mutein has an amino acid sequence identity of at least 70% with the wildtype T7 RNA polymerase sequence as set forth in SEQ ID NO:1.

14. The T7 RNA polymerase mutein of claim 12, wherein the mutein has the amino acid sequence set forth in any one of SEQ ID NOS: 2-6 or a functional fragment thereof.

15. A nucleic acid molecule, comprising a nucleotide sequence encoding a T7 RNA polymerase mutein, said mutein comprising a T7 RNA polymerase or a functional fragment thereof having at least one amino acid sequence mutation at a mutated amino acid sequence position compared to a wildtype amino acid at a corresponding wildtype amino acid sequence position in a wildtype T7 RNA polymerase, wherein either or both of:
(i) the mutated amino acid sequence position corresponds to amino acid sequence position 425 of SEQ ID NO:1 and the amino acid sequence mutation is selected from cysteine and tryptophan, and
(ii) the mutated amino acid sequence position corresponds to amino acid sequence position 441 of SEQ ID NO:1 and the amino acid sequence mutation is selected from valine, leucine and tyrosine.

16. A vector or a phagemid vector that comprises the nucleic acid molecule of claim 15.

17. A host cell, comprising the nucleic acid molecule of claim 15.

18. A method for producing a T7 RNA polymerase mutein, comprising:
culturing a prokaryotic or eukaryotic host cell that comprises a vector which comprises a nucleic acid molecule which comprises a nucleotide sequence encoding a T7 RNA polymerase mutein, said mutein comprising a T7 RNA polymerase or a functional fragment thereof having at least one amino acid sequence mutation at a mutated amino acid sequence position compared to a wildtype amino acid at a corresponding wildtype amino acid sequence position in a wildtype T7 RNA polymerase, wherein either or both of: (i) the mutated amino acid sequence position corresponds to amino acid sequence position 425 of SEQ ID NO:1 and the amino acid sequence mutation is selected from cysteine and tryptophan, and (ii) the mutated amino acid sequence position corresponds to amino acid sequence position 441 of SEQ ID NO:1 and the amino acid sequence mutation is selected from valine, leucine and tyrosine,
under conditions that allow expression of the encoded T7 RNA polymerase mutein by cultured cells; and
recovering the expressed T7 RNA polymerase mutein from the cultured cells.

19. A method for synthesizing a 2′-modified RNA molecule, comprising contacting a template nucleic acid with 2′-modified ribonucleotides and with a T7 RNA polymerase mutein, under conditions that allow synthesis of a 2′-modified RNA molecule by polymerase activity of the T7 RNA polymerase mutein,
wherein the mutein comprises a T7 RNA polymerase or a functional fragment thereof having at least one amino acid sequence mutation at a mutated amino acid sequence position compared to a wildtype amino acid at a corresponding wildtype amino acid sequence position in a wildtype T7 RNA polymerase, wherein either or both of: (i) the mutated amino acid sequence position corresponds to amino acid sequence position 425 of SEQ ID NO:1 and the amino acid sequence mutation is selected from cysteine and tryptophan, and (ii) the mutated amino acid sequence position corresponds to amino acid sequence position 441 of SEQ ID NO:1 and the amino acid sequence mutation is selected from valine, leucine and tyrosine.

20. The method of claim 19, wherein one, two or all three of:
(a) the 2′-modified ribonucleotides are selected from the group consisting of 2′-methoxy adenosine triphosphate (2′-methoxy ATP), 2′-methoxy guanosine triphosphate (2′-methoxy GTP), 2′-methoxy uracil triphosphate (2′-methoxy UTP) and 2′-methoxy cytosine triphosphate (2′-methoxy CTP) or combinations thereof;
(b) the 2′-modified ribonucleotides are 2′-methoxy modified ribonucleotides; and
(c) the 2′-modified RNA molecule is an RNA aptamer, a ribozyme, an siRNA, an miRNA or an antisense RNA.

21. An RNA molecule that comprises one or more 2′-modified ribonucleotides units, wherein said RNA molecule has a length of more than 100 nucleotides and is obtained by a method that comprises:
(a) contacting a template nucleic acid with 2′-modified ribonucleotides and with a T7 RNA polymerase mutein, under conditions that allow synthesis of a 2′-modified RNA molecule by polymerase activity of the T7 RNA polymerase mutein,
wherein the mutein comprises a T7 RNA polymerase or a functional fragment thereof having at least one amino acid sequence mutation at a mutated amino acid sequence position compared to a wildtype amino acid at a corresponding wildtype amino acid sequence position in a wildtype T7 RNA polymerase, wherein either or both of: (i) the mutated amino acid sequence position corresponds to amino acid sequence position 425 of SEQ ID NO:1 and the amino acid sequence mutation is selected from cysteine and tryptophan, and (ii) the mutated amino acid sequence position corresponds to amino acid sequence position 441 of SEQ ID NO:1 and the amino acid sequence mutation is selected from valine, leucine and tyrosine.


SEQUENCE LISTING (25 sequences)

SEQ ID NO: 1 - protein, 883 aa, Bacteriophage T7
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 2 - protein, 883 aa, Artificial
  Bacteriophage T7 DNA-dependent RNA polymerase mutein
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGCVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 883 aa, Artificial
  Bacteriophage T7 DNA-dependent RNA polymerase mutein
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGWVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - protein, 883 aa, Artificial
  Bacteriophage T7 DNA-dependent RNA polymerase mutein
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTVGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 5 - protein, 883 aa, Artificial
  Bacteriophage T7 DNA-dependent RNA polymerase mutein
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTLGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 6 - protein, 883 aa, Artificial
  Bacteriophage T7 DNA-dependent RNA polymerase mutein
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTYGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 7 - DNA, 36 bp, Artificial
  Amplification primer
aaaaaaaaaaaagtcgacttacgcgaacgcgaagtc

SEQ ID NO: 8 - DNA, 37 bp, Artificial
  Amplification primer
aaaaaaaaaaaaaagcttactgaacacgattaacatc

SEQ ID NO: 9 - DNA, 32 bp, Artificial
  strand of artificial double-strand fragment
aaaagacgtcaaactcgagaaagacgtcaaaa

SEQ ID NO: 10 - DNA, 32 bp, Artificial
  strand of artificial double-strand fragment
ttttgacgtctttctcgagtttgacgtctttt

SEQ ID NO: 11 - DNA, 40 bp, Artificial
  Mutagenesis primer
caacatggactggcgcggtnnbgtttacgctgtgtcaatg

SEQ ID NO: 12 - DNA, 40 bp, Artificial
  Mutagenesis primer
cattgacacagcgtaaacvnnaccgcgccagtccatgttg

SEQ ID NO: 13 - DNA, 39 bp, Artificial
  Mutagenesis primer
gcaaggtaacgatatgaccnnsggactgcttacgctggc

SEQ ID NO: 14 - DNA, 40 bp, Artificial
  Mutagenesis primer
cgccagcgtaagcagtccsnnggtcatatcgttaccttgc

SEQ ID NO: 15 - DNA, 57 bp, Artificial
  Molecular beacon
gcgagatccaaaaaaaaaaaccatctcgccgaattcgccctatagtgagtcgtatta

SEQ ID NO: 16 - DNA, 17 bp, Artificial
  T7 promoter containing oligonucleotide
taatacgactcactata

SEQ ID NO: 17 - DNA, 18 bp, Artificial
  Amplification primer
aggcctctagactgcagc

SEQ ID NO: 18 - DNA, 19 bp, Artificial
  Amplification primer
cgtaacttgtggtatcgtg

SEQ ID NO: 19 - DNA, 40 bp, Artificial
  Oligodeoxynucleotide probe
cttctcctttgctagccatatgtatatctccttcttaaag

SEQ ID NO: 20 - DNA, 21 bp, Artificial
  Artificial oligonucleotide with pU at 5′ terminus
atactcatggtcatagctgtt

SEQ ID NO: 21 - DNA, 19 bp, Artificial
  Amplification primer
aacagctatgaccatgagt

SEQ ID NO: 22 - DNA, 40 bp, Artificial
  Amplification primer
ctttaagaaggagatggatccgtggctagcaaaggagaag

SEQ ID NO: 23 - DNA, 17 bp, Artificial
  Amplification primer
caggaaacagctatgac

SEQ ID NO: 24 - DNA, 2652 bp, Bacteriophage T7
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaagacaaggctcgcaagtctcgccgtatcagccttgagttcatgcttgagcaagccaataagtttgctaaccataaggccatctggttcccttacaacatggactggcgcggttcgtgtttacgctgtgtcaatgttcaacccgcaaggtaacgatatgaccaaaggacgtcttacgctggcgaaaggtaaaccaatcggtaaggaaggttactactggctgaaaatccacggtgcaaactgtgcgggtgtcgataaggtttcgtttcctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctgcaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatccgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 25 - DNA, 112 bp, Artificial Sequence
  Aptamer Template
gataatacgactcactataggcgctccgaccttagtctctgtgccgctataatgcacggatttaatcgccgtagaaaagcatgtcaaagccggaaccgtgtagcacagcaga